(19) United States
(12) Patent Application Publication
(10) Pub. No.: US 2026/0174325 A1
(43) Pub. Date: Jun. 25, 2026

(54) METHOD FOR WIDE FIELD SELF-REFERENCED INTERFERENCE OCT CORNEA IMAGING

(71) Applicant: University of Miami, Miami, FL (US)

(72) Inventor: Marco Ruggeri, Miami, FL (US)

(21) Appl. No.: 19/430,472

(22) Filed: Dec. 23, 2025

Related U.S. Application Data
(60) Provisional application No. 63/737,887, filed on Dec. 23, 2024.

(51) Int. Cl. A61B 3/107; A61B 3/00; A61B 3/10; G02B 3/02

(52) U.S. Cl. CPC A61B 3/107; A61B 3/0025; A61B 3/102; G02B 3/02

(57) ABSTRACT

Various embodiments are presented for a method of wide-field self-referenced interference for OCT imaging, that allows for a focus on the cornea over a large diameter while reducing the number of artifacts in the signal. To accomplish this, the method includes operating a super luminescent diode to emit a light, using that light to illuminate an interferometer, collimating the light, and coupling the collimated light to an optical scanner and lens to produce a probing beam. The method also includes directing the beam through the lens onto the anterior corneal surface of the cornea and creating an incident angle between the probing beam and the anterior corneal surface. Finally, the method includes using the lens to ensure the beam is perpendicular to the corneal surface with the incident angle at less than two degrees, focusing from the center to the periphery of the corneal surface, aligning the beam with the reflective corneal surface to generate a self-referencing signal, and using the self-referencing signal to generate self-referenced interference. The self-referenced interference is detected with a spectrometer to generate a high-resolution image of the cornea over a large diameter while reducing the number of reflection artifacts experienced in typical OCT system. Additionally, a hybrid dual-path and self-referenced imaging method is disclosed in which an optical shutter in the reference arm enables sequential acquisition of conventional OCT and localized self-reference apex imaging, followed by fusion into a composite artifact-free OCT image.

### CROSS REFERENCE TO RELATED APPLICATIONS

This application claims priority to and the benefit of U.S. Provisional Application No. 63/737,887, filed Dec. 23, 2024, the entire content of which is hereby incorporated by reference herein.

### GOVERNMENT LICENSE RIGHTS

This invention was made with government support under R01EY033429 awarded by The National Institutes of Health. The government has certain rights in the invention.

### FIELD

The present inventive concepts relate to Optical Coherence Tomography technology, particularly utilizing a perpendicular beam on the highly reflective anterior corneal surface to generate a reference signal used to image the eye.

### BACKGROUND

Optical Coherence Tomography (“OCT”) is a powerful tool in ophthalmology, offering non-invasive, high-resolution imaging of ocular tissues, including the cornea. Visualizing and quantifying the thin layers of the cornea is essential for diagnosing and monitoring corneal diseases, assessing surgical outcomes, and guiding therapeutic interventions.

Conventional OCT systems for corneal imaging face challenges in achieving high resolution at the cornea periphery. These systems use a telecentric scan, where the beam is parallel to the eye's visual axis. In the periphery, the beam becomes defocused and tilted with respect to the corneal surface due to the cornea's steep curvature, resulting in decreased signal intensity and resolution that affects visibility of the corneal layers. Additionally, the cornea's steep curvature leads to the optical path of the OCT beam varying considerably from the center to the periphery, complicating the imaging of the entire cornea within the depth range of high-resolution spectral domain (SD) OCT systems, which is typically 2-3 mm.

Alternative OCT scanning strategies have been developed that rely on the beam remaining nearly perpendicular to and focused on the cornea across the corneal diameter. This has the advantage of improving the visibility of corneal layers over a large area and reducing the optical path difference of the scanning beam, which allows for imaging of larger corneal regions within the systems'depth range. However, a persistent issue across all scanning architectures is the strong reflection at the air-tear film interface at the locations where the beam is fully perpendicular to the cornea, such as the corneal apex with telecentric scanning system and potentially additional location in scanning system where the beam is converging to a point behind the cornea. This reflection generates a high-intensity signal that saturates the OCT detector when combined with the reference light in dual-path interferometers and produces a central vertical line artifact that interferes with the visualization of the underlying corneal structures.

Thus, despite attempts to produce an OCT system that allows the user to focus on the cornea across a large diameter while reducing the number of artifacts in the signal, these attempts have been unsuccessful overall.

### SUMMARY

The following is a brief summary of subject matter that is described in greater detail herein. This summary is not intended to be limiting as to the scope of the claims.

The general inventive concepts are based, in part, on the discovery that the corneal reflection can be leveraged as a reference signal for imaging of the cornea with optical coherence tomography (“OCT”) using a common path interferometer. In telecentric scanning architectures used in conventional OCT systems, the angle between the cornea and the incident beam becomes increasingly steep at the periphery resulting in minimal signal returned. In contrast, delivering the beam perpendicular to the cornea enables uniform collection of signal across the entire cornea. However, when using dual path interferometer used in conventional OCT systems, this approach might saturate the detector due to increased signal intensity. This approach employs a common path interferometer to enable self-reference interference between the highly reflective anterior corneal surface and the underlying structures across a wide field of view, enabled by a custom scanning lens that maintains near-perpendicular beam incidence on the cornea. This technique eliminates the need for an auxiliary reference arm and inherently avoids the central vertical line artifacts common in dual-path systems. Applicants have demonstrated this technique as a viable potential tool for early detection of cornea changes in patients suffering from keratoconus.

The general inventive concepts contemplate a method of wide-field self-referenced interference for OCT imaging. The method allows for a focus on the cornea over a large diameter while reducing the number of artifacts in the signal. To accomplish this, the method includes operating a super luminescent diode to emit a light, using that light to illuminate an interferometer, collimating the light, and coupling the collimated light to an optical scanner and lens to produce a probing beam. The method also includes directing the beam through the lens onto the anterior corneal surface of the cornea and creating an incident angle between the probing beam and the anterior corneal surface. Finally, in certain exemplary embodiments, the method includes using the lens to ensure the beam is perpendicular to the corneal surface with the incident angle at less than two degrees, focusing from the center to the periphery of the corneal surface, aligning the beam with the reflective corneal surface to generate a self-referencing signal, and using the self-referencing signal to generate self-referenced interference. The self-referenced interference is detected with a spectrometer which is used to create a high-resolution image of the cornea over a large diameter while reducing the number of reflection artifacts experienced in typical OCT system.

Additionally, various embodiments are presented for a hypercentric lens that allows for a beam to converge to a point that enables self-referenced interference. To accomplish this, the hypercentric lens includes a wide field lens with a large aperture that allows for a wide field of imaging, as well as one or more achromatic lenses. At least one achromatic lens is designed to expand any beam of light passing through it to fill the entire aperture of the wide field lens. The wide field lens is positioned closer to the cornea than the achromatic lenses. The wide field lens may feature an aspheric surface designed to match the anterior corneal curvature, enabling precise delivery of a beam perpendicular to the cornea. The wide field lens may have an optimal focus at a working distance that allows the beam of light to align perpendicularly to the cornea.

In certain exemplary embodiments, the general inventive concepts provide method that combines dual-path interferometry and self-referenced common-path interferometry (e.g., a hybrid imaging method) using an optical shutter disposed in a reference arm of the interferometer. In certain exemplary embodiments, this hybrid configuration comprises acquiring a first OCT image with the optical shutter in an open state to operate the system in conventional dual-path mode, followed by acquisition of a second OCT image with the optical switch in the closed state to operate the system in self-referenced common-path mode. In certain exemplary embodiments, the second acquisition follows in rapid succession to the first acquisition. The second image is generated within a localized region near the corneal apex where the incident beam is substantially perpendicular and sufficiently strong to generate self-interference. A composite OCT image is then formed by retaining peripheral image content from the dual-path image and replacing the apex region with corresponding content from the self-referenced image. This method suppresses the specular apex reflection artifact while preserving true curvature and depth geometry of the dual-path OCT image.

The above summary presents a simplified summary in order to provide a basic understanding of some aspects of the systems and/or methods discussed herein. This summary is not an extensive overview of the systems and/or methods discussed herein. It is not intended to identify key or critical elements or to delineate the scope of such systems and/or methods. Its sole purpose is to present some concepts in a simplified form as a prelude to the more detailed description that is presented later.

### BRIEF DESCRIPTION OF THE FIGURES

FIG. 1 shows a schematic of the wide-field self-referencing OCT system according to the general inventive concepts.

FIG. 2 shows a schematic of an additional embodiment of the wide-field self-referencing OCT system according to the general inventive concepts.

FIG. 3A shows a diagram of the position of the cornea with respect to the converging OCT scanning rays.

FIG. 3B shows a diagram of the position of the cornea with respect to the focus of the OCT beams.

FIG. 4A shows an OCT image acquired with the interferometer in conventional dual-path mode.

FIG. 4B shows an OCT image acquired with the interferometer in common-path mode according to the general inventive concepts.

FIG. 5 is a detailed inset of the galvanometer and lens.

FIG. 6 shows the results of the self-referencing common-path mode in the left and right eyes of two different subjects when assessed using the systems and methods of the general inventive concepts.

FIG. 7 shows a map of the corneal microlayers thickness in the left and right eyes of two different subjects when assessed using the systems and methods of the general inventive concepts.

FIG. 8 shows a map of the incident angles of the OCT beam to the cornea across the field of the eye.

FIG. 9A is an image showing a wide-field OCT image of a normal cornea.

FIG. 9B is an image of a magnified view of a region immediately anterior and posterior to Bowman's layer, obtained by averaging three consecutive radial OCT images.

FIG. 10 is a flow chart illustrating a method for wide field self-referenced interference OCT imaging of the cornea according to the general inventive concepts.

FIG. 11 is a flow chart illustrating a method for suppressing corneal apex reflection artifacts in an OCT image according to the general inventive concepts.

FIG. 12 is a series of images showing apex artifact correction, demonstrated on a zoomed-in central portion of a wide-field corneal OCT image. The top row shows dual-path OCT data and self-referenced OCT data undergo apex artifact localization, lateral alignment, boundary detection, saturated region removal, axial alignment, and fusion to generate an artifact-free composite apex region. Bottom row shows corresponding self-referenced OCT images used for alignment and replacement. Only the central apex region is shown here for clarity; in the full wide-field image, this region represents a small fraction of the lateral scan extent. The final composite image preserves the original dual-path axial depth geometry outside the apex region while eliminating saturation at the corneal apex.

### DETAILED DESCRIPTION

Various technologies pertaining to a wide-field self-referencing OCT method are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details. Further, it is to be understood that functionality that described as being carried out by certain system components may be performed by multiple components. Similarly, for instance, a component may be configured to perform functionality that is described as being carried out by multiple components.

Unless defined otherwise, all technical and scientific terms used herein have the same meaning as commonly understood by one of ordinary skill in the art to which the invention belongs.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made. All references to methods or processes are intended to comprise and encompass each embodiment of the compositions described herein, including those described in the examples.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all subranges between (and inclusive of) the minimum value of 1 and the maximum value of 10; that is, all subranges beginning with a minimum value of 1 or more (e.g., 1 to 6.1), and ending with a maximum value of 10 or less (e.g., 2.3 to 9.4, 3 to 8, 4 to 7), and finally to each number 1, 2, 3, 4, 5, 6, 7, 8, 9, and 10 contained within the range.

The systems and corresponding methods of the present disclosure can comprise, consist of, or consist essentially of the essential elements and limitations of the disclosure as described herein, as well as any additional or optional ingredients, components, or limitations described herein or otherwise useful in the general inventive concepts.

To the extent that the terms “include,” “includes,” or “including” are used in the specification or the claims, they are intended to be inclusive in a manner similar to the term “comprising” as that term is interpreted when employed as a transitional word in a claim. Furthermore, to the extent that the term “or” is employed (e.g., A or B), it is intended to mean “A or B or both A and B.” When the Applicant intends to indicate “only A or B but not both,” then the term “only A or B but not both” will be employed. Thus, use of the term “or” herein is the inclusive, and not the exclusive use. In the present disclosure, the words “a” or “an” are to be taken to include both the singular and the plural. Conversely, any reference to plural items shall, where appropriate, include the singular.

In some aspects, it may be possible to utilize the various inventive concepts in combination with one another. Additionally, any particular element recited as relating to a particularly disclosed embodiment should be interpreted as available for use with all disclosed embodiments, unless incorporation of the particular element would be contradictory to the express terms of the embodiment. Additional advantages and modifications will be readily apparent to those skilled in the art. Therefore, the disclosure, in its broader aspects, is not limited to the specific details presented therein, the representative composition, or the illustrative examples shown and described. Accordingly, departures may be made from such details without departing from the spirit or scope of the general inventive concepts.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Conventional attempts have sought to increase the field of view of corneal OCT imaging while obtaining high resolution images of the corneal layers using dual path interferometers. Many efforts revolved around generating high resolution images of the cornea with sufficient signal from the center to the periphery; using common path OCT provides the ancillary advantage that the artifact is not present in the image. In one example, found in U.S. Pat. No. 8,864,309, OCT beams are delivered perpendicularly to the cornea across a large field, though a reference arm and mirrors are still necessary (dual path interferometer), and artifacts are still common. One publication, Pattan et al., utilized self-referenced OCM (optical coherence microscopy) imaging of the cornea using a common path interferometer in combination with parallel scanning beams (telecentric scan), which narrowed the field of vision and limited the lateral amount of cornea visible. In U.S. Pat. No. 10,045,692, the applicant also used a telecentric scan combined with self-referenced interference to generate OCT images, but for the retina, not the cornea, where the scanning architecture diverges significantly from that used for corneal imaging. Beer et al. used an off-the-shelf optic lens to generate converging beams that were nearly perpendicular to the cornea utilizing a classic OCT imaging technique without self-reference interference by using a dual path interferometer. In Krstajic et al, there is a discussion of using a general tissue surface as the reference for generating self-interference in OCT imaging, but it does not disclose utilizing a perpendicular beam in conjunction with the tissue surface. Instead, the technique highlighted in Krstajic involves placing a diffuser, such as aluminum hydroxide powder, on the tissue to generate a sufficient reference signal to enable self-referenced interference and focuses on using the technique on tissues that are not naturally reflective, such as the skin, as opposed to the cornea. Other self-referenced interference methods used in the field similarly require external factors placed directly on the tissue surface to increase the reflectivity and achieve self-referenced interference; for example, placing a reflective element such as a glass window onto the tissue. The inventive concept described below avoids this type of external equipment.

All publications and patent applications identified herein are incorporated by reference in their entirety and to the same extent as if each individual publication or patent application was recited herein in their entirety.

In certain exemplary embodiments, the general inventive concepts contemplate a method of wide-field self-referenced interference for OCT imaging. The method allows for a focus on the cornea over a large diameter while reducing the number of artifacts in the signal. To accomplish this, the method includes operating a super luminescent diode to emit a light, using that light to illuminate an interferometer, collimating the light, and coupling the collimated light to an optical scanner and lens to produce a probing beam. The method also includes directing the beam through the lens onto the anterior corneal surface of the cornea and creating an incident angle between the probing beam and the anterior corneal surface. Finally, in certain exemplary embodiments, the method includes using the lens to ensure the beam is perpendicular to the corneal surface with the incident angle at less than two degrees, focusing from the center to the periphery of the corneal surface, aligning the beam with the reflective corneal surface to generate a self-referencing signal, and using the self-referencing signal to generate self-referenced interference. The self-referenced interference is detected with a spectrometer which is used to create a high-resolution image of the cornea over a large diameter while reducing the number of reflection artifacts experienced in typical OCT system.

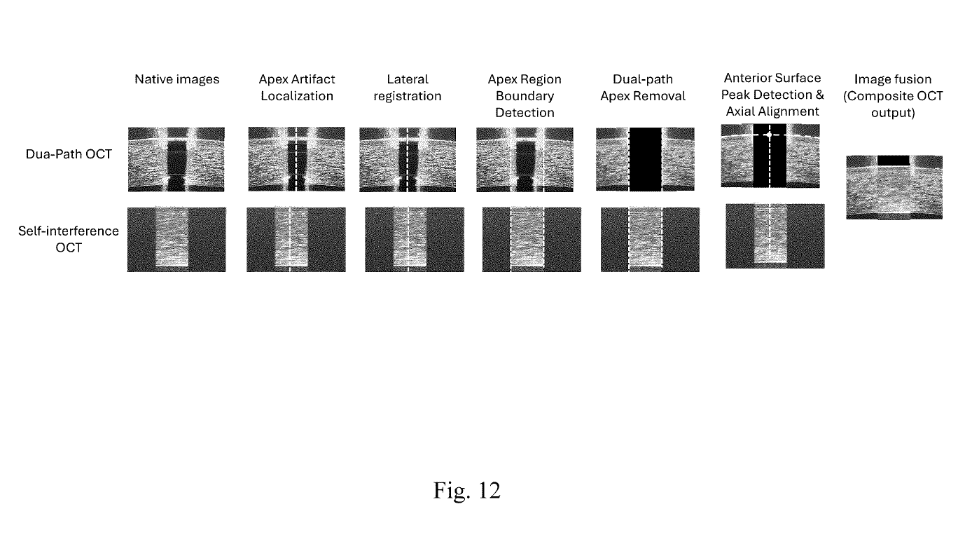

In certain exemplary embodiments, the general inventive concepts also provide a method that combines dual-path interferometry and self-referenced common-path interferometry (e.g., a hybrid imaging method) using an optical shutter disposed in a reference arm of the interferometer In certain exemplary embodiments, this hybrid configuration comprises acquiring a first OCT image with the optical shutter in an open state to operate the system in conventional dual-path mode, followed by acquisition of a second OCT image with the optical switch in the closed state to operate the system in self-referenced common-path mode. In certain exemplary embodiments, the second acquisition follows in rapid succession to the first acquisition. The second image is generated within a localized region near the corneal apex where the incident beam is substantially perpendicular and sufficiently strong to generate self-interference. A composite OCT image is then formed by retaining peripheral image content from the dual-path image and replacing the apex region with corresponding content from the self-referenced image. This method suppresses the specular apex reflection artifact while preserving true curvature and depth geometry of the dual-path OCT image. In certain exemplary embodiments, the second OCT image is generated only when an incident beam is substantially perpendicular to the corneal apex. In certain exemplary embodiments, the composite OCT image preserves dual-path axial depth geometry outside the apex region In certain embodiments, the corneal apex region comprises about 10-20% of the lateral scan (e.g., approximately 1 mm of a 10 mm-wide corneal scan), such that the dual-path axial depth geometry is preserved over at least about 80% of the image outside the apex region. See FIG. 12 for an exemplary embodiment. In certain embodiments, the region outside the apex area is still the majority (>80%) of the composite image. According to the general inventive concepts, the “apex region” is intended to be a relatively small blending zone, not a predetermined fraction of the field. In practice the corneal apex region corresponds to and is based on a portion of the scan width and the corneal curvature. For a typical wide-field corneal scan of about 8-12 mm chord length, the apex region over which self-referenced data are substituted extends over roughly -0.5-1.5 mm centered on the apex, so on the order of 5-15% of the lateral field of view, corresponding to a similar fraction of the image area. The remaining ˜85-95% of the image preserves the dual-path axial depth geometry.

In certain exemplary embodiments, the optical shutter comprises a mechanical shutter, electro-optic modulator, acousto-optic modulator, or MEMS-based optical shutter. In certain exemplary embodiments, the first and second OCT images are acquired sequentially within less than 100 milliseconds. In certain embodiments, the first and second OCT images are acquired sequentially. In certain embodiments, the image acquisition comprises a time interval short enough that motion of the subject's eye remains within an image-registration tolerance. In certain exemplary embodiments, the acquisition interval between first and second OCT images is less than about 200 milliseconds, including about 200 milliseconds to about 50 milliseconds, about 150 milliseconds to about 50 milliseconds, about 100 milliseconds to about 50 milliseconds, and including less than about 50 milliseconds. The general inventive concept is not intended to be limited to specific time gap values. Rather, the purpose of a short time gap is to minimize relative motion of the eye between the dual-path and self-referenced acquisitions so that the two images can be accurately registered and blended. However, in larger time gaps, image registration becomes less robust as the interval increases (due to fixational eye movements, blinking, etc.). Important factors for determining a suitable interval include that the time between acquisitions is short enough that motion remains within the registration tolerance of the system, which depends on scan pattern, A-scan rate, and patient cooperation.

In certain exemplary embodiments, the composite OCT image eliminates vertical saturation artifacts while preserving structural detail and boundary definition of all corneal layers, including the epithelium, Bowman's layer, and stromal tissue.

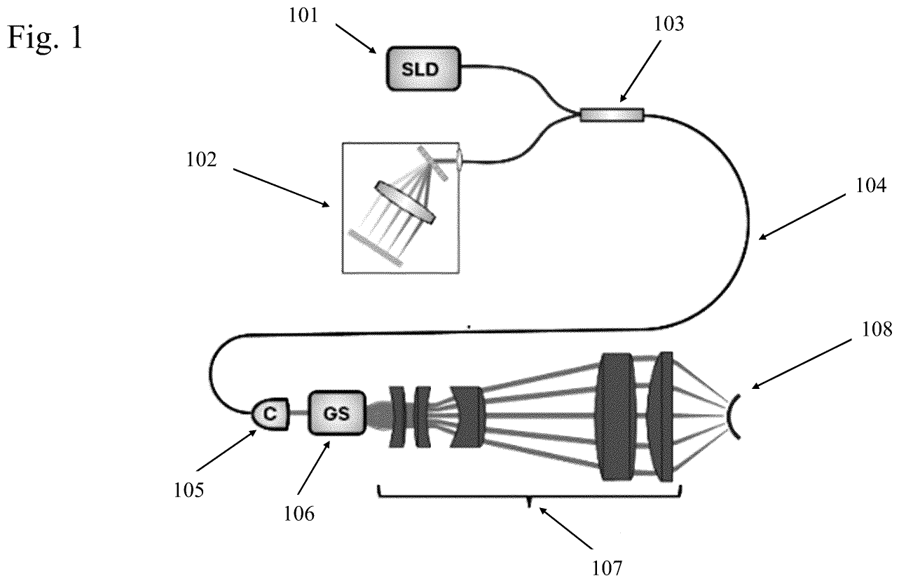

Referring now to FIG. 1, there is shown a schematic of the wide-field self-referencing OCT system according to the general inventive concepts. The wide-field self-referencing system includes a light source 101. In certain embodiments, the light source comprises a super luminescent diode. In certain embodiments the light source is operated to emit light. In certain embodiments, the light source is operated to emit light at a central wavelength of 850 nm and providing a resolution of 2.7 um in air. However, a person of ordinary skill in the art will recognize that OCT systems can function in both the visible and near infrared spectrum, and as such the inventive method applies to any OCT system developed within those ranges (e.g., between 400 nm and 2500 nm, including 500 nm to 2000 nm, including 550 nm to 1700 nm, including 600 nm to 1500 nm, including 675 nm to 1250, including 700 nm to 1050 nm, including 740 nm to 925 nm, including 800 nm to 875 nm). The light from the diode illuminates an interferometer 103, which, in certain embodiments may be e.g., a fiber optic coupler, a fiber optic circulator, or a free space interferometer. The light then passes into a sample arm 104, such as a fiber cable or a free space arm. After exiting the sample arm 104, the light passes through a collimator 105 that collimates the light. The collimated light then passes through a dual axis galvanometer scanner 106 to produce a probing beam. In other embodiments, however, other types of optical scanners, such as MEMS scanners, polygon scanners, acousto-optic deflectors, electro-optic scanners, piezoelectric scanners, resonant scanners, and liquid crystal optical scanners, may also be used. After passing through the galvanometer scanner 106, the beam of light is directed through a lens 107. As can be seen from the figure, the term “lens” does not necessarily refer to one single device, but, in certain embodiments, may refer to a series or system of a plurality of lenses that operate to expand and direct the light in accordance with the general inventive concepts. In certain embodiments, the lens 107 is hypercentric (as shown in the figure). The hypercentric lens 107 is configured to converge the scanning rays to a point positioned behind the cornea 108. Because the lens 107 is hypercentric, it features negative field curvature, such that the image plane curves away from the lens. The hypercentric lens 107 is used to create an incidence angle formed between the OCT beam and the cornea; specifically, is the angle formed between the OCT beam's direction and the direction fully perpendicular to the corneal surface at a specific location (i.e., the anterior corneal structure). In the experiments associated with the inventive method described herein, it was determined that an incidence angle of approximately two degrees or less provides sufficient reflected reference signal from the cornea to generate self-referenced interference. Depending on illumination power, beam geometry, detector sensitivity, and other system design parameters, suitable incidence angles may extend beyond two degrees, for example up to approximately 4-5 degrees or more in certain embodiments. Acceptable angular tolerance depends on multiple system parameters including illumination power, detection sensitivity, beam geometry on the cornea, and optical numerical aperture. In certain embodiments, the systems and methods comprise an incidence angle of less than 4°, including from about 0° (i.e., full orthogonality), to about 3°, including about 0.5° to about 3°, including about 1° to about 2°.

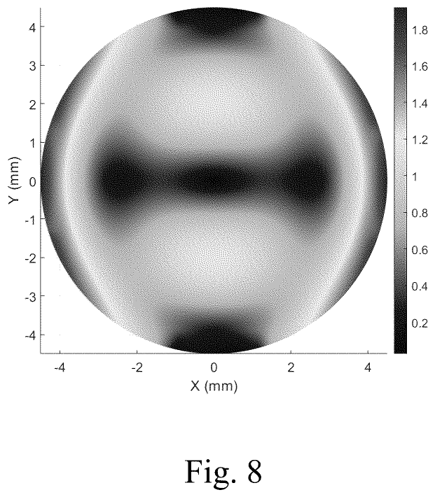

Referring now to FIG. 8, there is shown a map of the incident angles of the OCT beam across the field of the eye as used to generate the self-referenced interference. As illustrated by FIG. 8, the incident angle of the OCT beam across the cornea is very small (less than 2 degrees) along the perpendicular direction of the cornea, and though the angle still increases along the edges, it is still at an incidence angle of approximately two degrees or less (i.e. approximately full perpendicularity). This low incident angle reduces the artifacts produced in traditional methods and enables the self-interference as discussed herein.

Referring again to FIG. 1, as the hypercentric lens 107 is utilized to ensure the probing beam remains substantially perpendicular to the anterior cornea surface, it must necessarily move through near perpendicularity (i.e. with an incident angle of no more than five degrees, but greater than the two degrees necessary to achieve full perpendicularity) to the anterior corneal surface. This stage allows the user to optimize the signal to noise ratio (“SNR”) by shifting the focus from the center to the periphery of the cornea. The illustrated embodiment shows the hypercentric lens 107 including an aspheric surface used near the eye in order to match the curvature of the anterior corneal shape, which allows for large field imaging while minimizing spherical aberrations. The hypercentric lens 107 also includes one or more achromatic lenses adapted to expand the beam while reducing chromatic aberrations. The expanded beam is able to fill the aperture of the final lens, enabling scanning of a wide region.

A person of ordinary skill in the art, however, will recognize that a hypercentric lens with an aspheric surface is not the only lens design that could work; there is no single design approach for this type of lens. Those of ordinary skill in the art will recognize that a wide variety of optical recipes may be suitable and still fall within the general inventive concepts, albeit with varying degrees of success. Currently, comparing optical designs using optical simulators is not sufficient to predict OCT image quality and comparing optical designs experimentally is impractical due to the high costs of developing and testing multiple designs. Current simulations suggest that other designs are suboptimal but could achieve similar results, as this imaging application may tolerate variations. For example, as stated above, in the illustrated embodiments an aspheric element was included in the hypercentric lens to better align the beam with the cornea's naturally aspheric shape. The aspheric lens was chosen to improve the OCT image quality as suggested by optical simulations. However, in other embodiments, the hypercentric lens includes a spherical surface or free-form surfaces. In an additional embodiment, a single lens with a single aspheric surface is used. Again, due to the flexible nature of the inventive method, although these lenses may be suboptimal, they may still achieve suitable, though suboptimal, results.

In a traditional OCT system for corneal imaging (usually SD-OCT or SS-OCT, also known as Fourier domain OCT) a dual-path approach is frequently used. With a dual-path approach, the reference and sample arms are separate, and each arm has a dedicated light path. Light is split between these two paths, with one directed toward the sample and the other toward a stationary reference mirror.

Another type of approach is known as a common-path approach. In a common-path approach, the sample and reference paths are combined, and a reflective interface within or in contact with the sample (in this case, the corneal surface) serves as the reference. In the illustrated embodiments, a common path approach is used. After the beam is aligned with the anterior corneal surface of the cornea 108, the light hits the anterior corneal surface of the cornea 108 and a portion is reflected back in the interferometer generating the reference signal. Simultaneously, light backscatters from the internal tissue structure (i.e., within the cornea 108). These backscattered and reference light signals recombine, producing interference patterns.

The fiber coupler within the interferometer 103 directs part of the backscattered and reference light into a detection arm connected to a spectrometer 102, which provides an imaging depth. In certain embodiments, the spectrometer detects the interference in the spectral domain and defines the imaging depth based on its spectral resolution. In certain embodiments, the spectrometer 102 provides an imaging depth of about 2.3 mm and rate of about 32,000 A-lines/second. Non-limiting embodiments of suitable spectrometers include the Envisu 2300 or Leica microsystems, though a person of ordinary skill in the art will appreciate that a variety of different options exist as technology develops in this space including Swept Source OCT (SS-OCT). Persons of ordinary skill in the at will also recognize that a range of spectrometers may also produce a range of imaging depths at differing rates, depending on the technology available to practitioners at the time. For example, in other embodiments the spectrometer 102 may provide an imaging depth between 0 mm and 40 mm, and the speed may vary from 1000 A-lines per second to 10,100 Mhz (wherein a Mhz is 1,000,000 A-lines per second).

The spectrometer 102 detects the interference pattern in the spectral domain that is generated when the backscattered light recombines with light from the reference path. The interference pattern collected by the spectrometer pattern is used to reconstruct the reflectivity depth-profile of the cornea, thereby allowing a series of cross-sectional OCT images of the cornea. In this way, the spectrometer generates an OCT image of the cornea. More specifically, interference data from the sample is detected across a spectrum (i.e., it is spectrometer-based) or with a swept laser (also known as swept-source), allowing the entire depth profile to be captured in one exposure. A Fourier transform may then convert the spectral data into depth-resolved images.

In a conventional OCT system, the second path of light (i.e., the reference path) is sent to a reference arm with a known length and one or more mirrors. However, in the illustrated embodiment, the corneal reflection itself has been leveraged as a reference signal for imaging of the cornea, which enables the self-referenced interferometry. This improves the final generated image of the system by removing the vertical line artifact typically found in conventional OCT systems, allowing for improved imaging. Because the common-path interferometer 103 utilizes a single optical path, the backscattered light from the cornea is combined with a reference reflection from the anterior cornea within the same optical path.

This approach enables self-interference between the air-cornea interface and underlying corneal structures, overcoming limitations of conventional OCT systems that typically rely on a dual path interferometer, particularly for imaging the anterior corneal layers up to the periphery. In the illustrated embodiments, the hypercentric lens 107 facilitates self-referenced OCT imaging across wider field, including about a 9 mm diameter zone, ensuring the beam is substantially perpendicular (with an incident angle less than two agrees), wherein the perpendicular beam is in alignment with the anterior cornea to generate a robust reference signal. Meanwhile, the common-path interferometer 103 uses the highly reflective anterior corneal surface as a reference, eliminating the central vertical line artifact seen in conventional dual-path OCT systems and the need for a separate reference arm. The general inventive concepts avoid dispersion mismatch and polarization fading by sharing the same optical path for both sample and reference light, simplifying system design. In certain exemplary embodiments, it also flattens the corneal image to the anterior surface maximizing the use of the SD-OCT axial range.

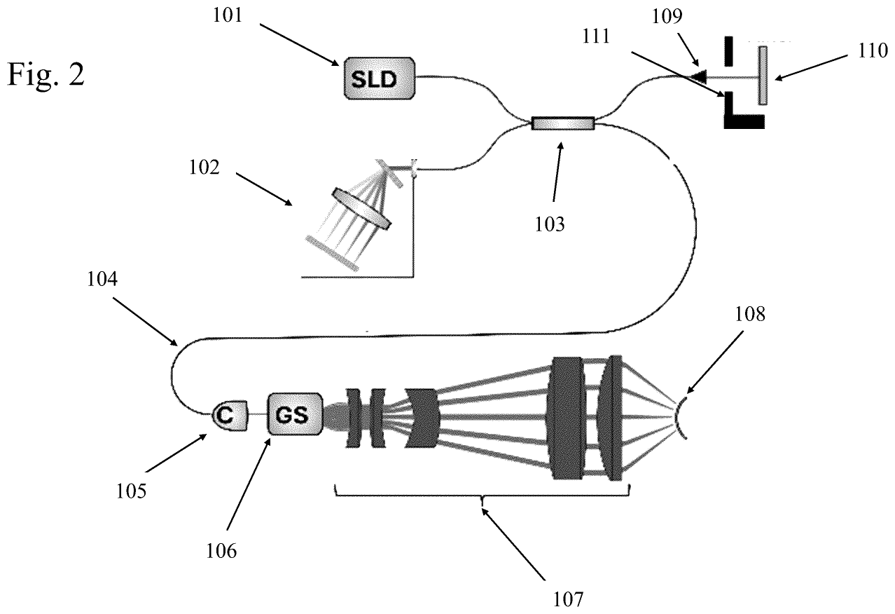

Referring now to FIG. 2, there is shown a schematic of the wide-field self-referencing OCT system. In certain embodiments of the wide-field self-referencing OCT system of the general inventive concepts, the interferometer 103 includes an optical shutter 111 and reference arm 109. The optical shutter 111 allows the interferometer to operate in two selectable configurations: a dual path mode (also known as Michelson mode) and a common path mode (also known as Fizeau mode).

A current challenge with self-referential imagery is the imaging process itself, as the real-time display only shows images when the incident beam is properly aligned, i.e., perpendicular to the corneal surface. However, this requires precise probe positioning and is initially difficult to position correctly, often requiring incremental adjustments until part of the image becomes visible and making the process time-consuming compared to conventional OCT systems that provide immediate feedback.

The illustrated embodiments seek to overcome these deficiencies, wherein the reference arm 109 of the interferometer 103 includes an optical shutter, allowing access to a mirror 110 for use as a reference. In certain embodiments, the shutter 111 is remotely controlled (e.g., by using a food pedal or other user interface). The general inventive concepts also contemplate the shutter 111 being automated.

In operation, when the optical shutter 111 is open, the interferometer 103 operates in dual path mode, and utilizes the mirror 110 attached to the reference arm 109 of the interferometer to generate the reference signal, similar to how a traditional dual-path OCT system would perform an assessment. In other words, the optical shutter 111 allows the mirror 110 to be displayed such that the fiber coupler in the interferometer 103 can split the light and send one path (i.e., the reference path of light) towards the mirror 110, such that the mirror 110 generates the reference signal.

The dual path mode allows for an image of the cornea to be displayed even if the beam is not perfectly perpendicular to the corneal surface, simplifying initial alignment and streamlining the overall process. Ideally, dual path mode is used for the initial alignment; and the mirror 110 provides a preliminary reference signal. Once the proper alignment is achieved, the shutter 111 can be closed to switch to common-path mode, where the anterior corneal surface 108 provides the reference signal for self-referenced interferometry. At this point, the beam is realigned to be substantially perpendicular to the cornea, and may be further refined by the operator to optimize the imaging process in a manner that would be evident to a person of ordinary skill in the art (e.g. producing micro adjustments to the alignment in order to generate the image with the fewest artifacts or distortions).

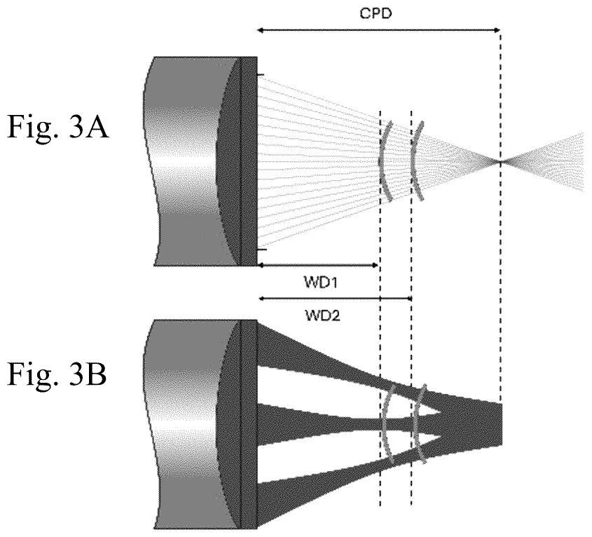

Referring now to FIGS. 3A and 3B, there is shown a diagram of the position of the cornea where the OCT imaging rays are focused (3A) and defocused (3B).

As seen in both images, a distance CPD is marked to convey the point where the rays converge. In the illustrated embodiment, the CPD is envisioned at 36 mm. In dual-path mode, the best position is a first working distance (WD1) away from the lens, because that is the distance where the single beams are focused on the cornea. An example of this is illustrated in FIG. 3B, where the focal “plane” is at WD1, which leads to high resolution. In the illustrated embodiment, WD1 is 25 mm. However, in dual-path mode, the lens could be positioned elsewhere along the path, and an image could still be generated, albeit with a lower resolution. At WD1, the scanning rays are nearly perpendicular (i.e., at an incident angle greater than two degrees) to the anterior cornea in order to optimize the signal to noise ratio across the corneal diameter. However, in dual-path mode, full perpendicularity (i.e., at an incident angle less than two degrees) is avoided in order to confine specular reflections to the apex. In the illustrated embodiment, the beam is focused on the cornea with a spot diameter of 16 um (FWHM) and the lateral field is 11 mm.

Full perpendicularity occurs at a second working distance (WD2) away from the lens. In common-path mode, an image can only be generated at this point, when the rays are fully perpendicular to the cornea. In the illustrated embodiment, WD2 is greater than WD1, in that it is located further away from the lens. In the illustrated embodiment, the second working distance is at approximately 28 mm. At this distance, the incidence angle is within 2 degrees over the entire field, enhancing reference signal collection. Various simulations were performed to determine the optimal working distance by minimizing the root-mean-square deviation of the incident ray angles. One example of such a simulation is provided in FIG. 8.

In dual-path mode, it is when nearing WD2—the only position where self-reference interference can be achieved—that the optical shutter may be closed to obtain a self-referenced image. In dual-path mode, WD2 is recognized by the saturation in the image due to strong reflections from both the mirror and anterior cornea. By blocking the reference arm at this point (i.e., closing the shutter), the anterior corneal reflection no longer saturates the detector, allowing for self-reference interference.

Referring to FIG. 3B specifically, the OCT beam is shown defocused at WD2. This is because the hypercentric lens was not originally designed to generate the smallest spot size when the beam is perpendicular to the cornea across its entire diameter. In fact, the hypercentric lens was originally designed with optimal focus at WD1, where the beams converge (as seen in FIG. 3A). As a result, in the associated experiments where the working distance was at WD2, the beam was defocused, resulting in a relatively large beam size at the cornea (62 μm FWHM). Ideally, a lens specifically designed for self-reference interference would have a focus at the same working distance where the beams align perpendicularly to the cornea.

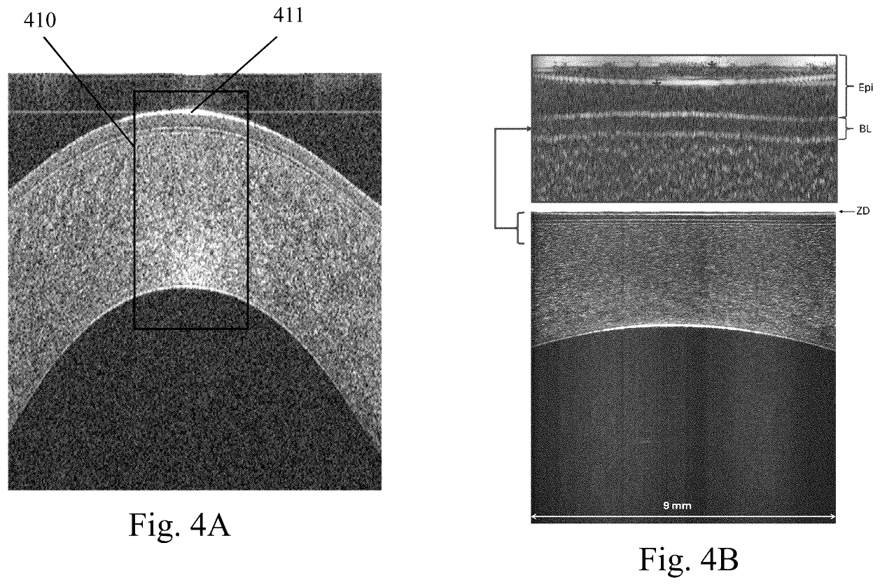

Referring now to FIGS. 4A and 4B, there is shown an OCT image acquired with the interferometer in conventional dual-path mode (4A) and common-path mode (4B), respectively. OCT data sets were acquired using a radial pattern with 10 degrees angle spacing in both configurations. Each image, consisting of 2000 A-lines, underwent two-fold averaging to improve SNR. The acquisition lasted 2 seconds, and the images were then segmented to map the thickness of the anterior corneal layers.

FIG. 4A shows an OCT image acquired with the interferometer in dual-path mode from a healthy 48-year-old subject, though the image is distorted slightly due to the scanning architecture. The epithelium (Epi) and Bowman's layer (BL) are visible across the corneal diameter (11 mm). Additionally, the basal epithelial layer (BE) 411 appears hypo-reflective in this image. However, as is common with this type of imaging, a vertical artifact, highlighted in the box marked 410, mars the image, making it difficult to view parts of the image in detail.

The common-path mode image, seen in FIG. 4B, was acquired from a healthy 27-year-old subject. There is no vertical line artifact, and the epithelial layers can be seen in detail. The posterior corneal surface appears bright, with hypercentric scanning improving visibility by minimizing the angle of incidence of the beam with the posterior cornea compared to telecentric scanning. Due to the common-path configuration with the anterior surface acting as a reference, any irregularity in the anterior corneal shape compounds to the observed shape of the posterior cornea. This approach intrinsically flattens the image to the corneal surface (i.e., air-tear film interface), in order to align it to the zero-delay reference (seen at the top of the image). Here, the epithelium and Bowman's layer are visualized in high resolution and extend across a 9 mm imaging field. However, image artifacts, primarily DC (direct current) noise near the zero-delay position, overlap with the anterior epithelium, which could be removed with background noise subtraction techniques. These artifacts, however, do not overlap with the Epithelium-Bowman's layer interface, allowing for epithelium thickness quantification but affecting resolution of substructures such as the basal epithelium.

Although the anterior cornea serves as a stable reference aligned with the zero delay, one drawback of the inventive method is that it remains vulnerable to motion artifacts. Lateral movements can cause signal loss due to the disruption of perpendicular beam alignment, in addition to causing a shift in image location. Axial movements result in changes to the scan width as the cornea shifts across the converging rays. However, re-opening the shutter to utilize the interferometer in dual-path mode allows the user to realign the beam as needed, should it become too disrupted.

The inventive system operates effectively within a specific range of corneal curvatures. However, one drawback is shape deviations from the designed corneal curvature (7.64 mm), such as in eyes with high astigmatism or keratoconus, which affect the system sensitivity generating shadowing effects on the image. This suggests that the method is better suited in the early stages of corneal diseases, where corneal curvature deviations are moderate. Dry eye conditions might also reduce corneal reflectance, further reducing system sensitivity.

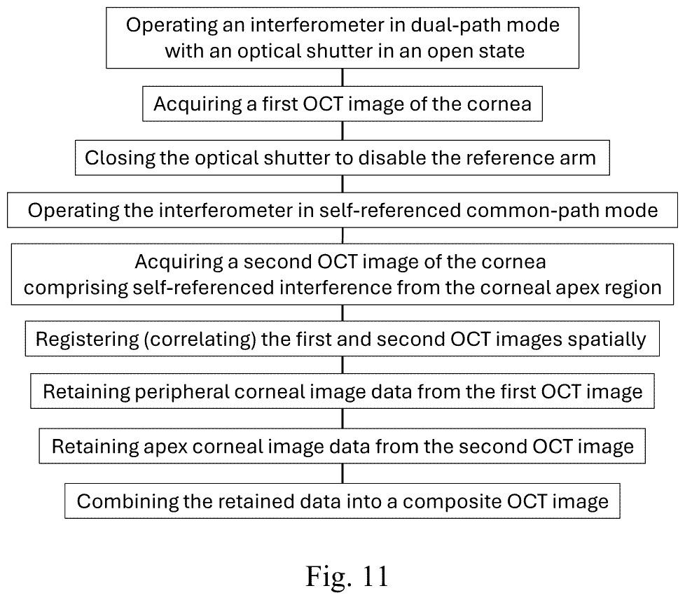

In certain embodiments, the general inventive concepts contemplate a hybrid acquisition method. In other words, the wide-field OCT system is operated in a hybrid acquisition mode. See FIG. 11, illustrating an exemplary embodiment of a hybrid acquisition method. The term hybrid acquisition mode refers to one that combines conventional dual-path interferometry with localized self-referenced common-path interferometry using an optical shutter disposed in a reference arm of the interferometer, as described in connection with the exemplary embodiments discussed above, in order to eliminate deleterious specular reflection artifacts occurring at the corneal apex while preserving the native geometric curvature of the cornea.

In conventional dual-path OCT imaging, when the probing beam is delivered near-perpendicularly to the corneal apex, a strong specular reflection is generated at the air-tear film interface. When this reflection is combined with the reference arm signal, it often produces saturation and a dominant vertical line artifact that obscures central corneal microstructures and compromises segmentation accuracy.

To overcome this limitation, an optical shutter is disposed in the reference arm of the interferometer. In a first acquisition state, the optical shutter is opened, and a first OCT image is acquired in conventional dual-path mode using a reference mirror. This first image provides high-quality corneal imaging across the peripheral cornea but contains a saturated apex artifact.

Subsequently, the optical shutter is closed to disable the reference arm and place the interferometer in a self-referenced common-path mode. In certain embodiments, the two images are acquired with substantially little or no delay between them; however, the actual interval is primarily governed by the response time of the optical shutter and overall system timing. Time intervals on the order of the shutter transition time, typically from tens of microseconds up to hundreds of milliseconds depending on shutter type and system configuration, may be used. While shorter intervals reduce the risk of eye motion between acquisitions, the method remains operable with longer intervals provided that sufficient image registration can be achieved. In this configuration, the only remaining reference signal is generated by reflection from the anterior corneal surface itself. Because self-referenced interference under near-perpendicular incidence is only generated in a localized region around the corneal apex, a second OCT image is produced that contains valid structural information only within the apex region.

The first and second OCT images are then spatially registered and combined into a composite OCT image. Registration and fusion are performed automatically by a computer processor without requiring manual region selection (see e.g., FIG. 12). In certain exemplary embodiments, image registration is carried out by first detecting the lateral center of the apex saturation artifact in the dual-path image and aligning it with the lateral center of the apex region in the self-referenced image. The saturated portion of the dual-path image is then removed or masked, and the anterior corneal surface in the remaining non-saturated dual-path data is detected and axially aligned to the zero-delay position defined by the self-referenced image. This alignment establishes accurate pixel correspondence between the two datasets. The apex region to be replaced may be identified using image-derived criteria such as saturation signatures in the dual-path image, intensity profile characteristics in the self-referenced image, or a combination thereof. In the resulting composite image, peripheral and mid-peripheral corneal regions, representing the majority of the lateral scan extent (for example, approximately 80-95%, depending on scan geometry), are retained from the dual-path acquisition, whereas the apex region, representing a relatively small fraction of the lateral extent (for example, approximately 5-20%), is replaced with artifact-free self-referenced apex data. This hybrid fusion preserves the true corneal curvature and axial depth geometry of the dual-path configuration while eliminating the apex saturation artifact without flattening the entire image to the zero-delay position.

This hybrid acquisition and fusion method enables full-field, curvature-preserving corneal OCT imaging without telecentric apex saturation artifacts, thereby improving segmentation of the epithelium, Bowman's layer, and posterior cornea while maintaining compatibility with conventional OCT image interpretation pipelines.

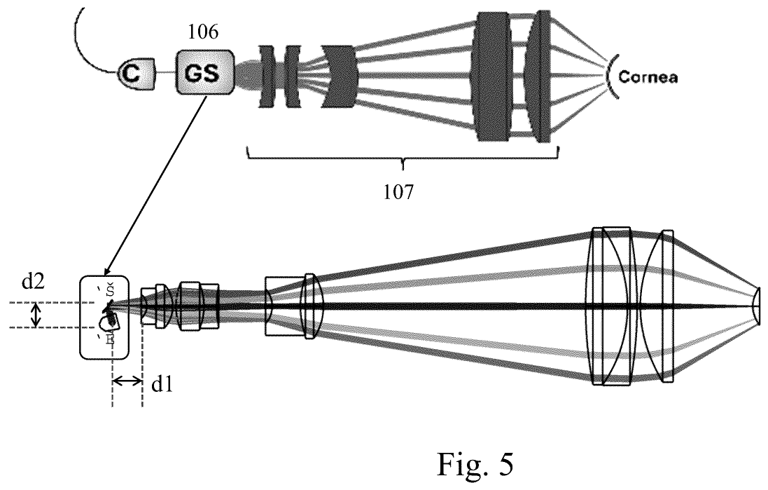

Referring now to FIG. 5, there is shown a more detailed inset of the galvanometer 106 and lens 107 and how modifications to the galvanometer 106 may affect the lens 107. In certain embodiments, the galvanometer 106 (and more specifically, the mirrors within it) are positionable, and may be adjusted to compensate for corneal curvature deviations across different corneal shapes such as those mentioned above. The galvanometer 106 is positioned a distance d1 from the lens 107. Adjusting the position d1 relative to the lens 107 is one means of reshaping the angle distribution of rays (i.e., the probing beam) incident on the cornea to achieve an image generated using the inventive method described but without the generation of shadowing effects on the image that persist when the cornea has some amount of shape deviation (e.g. astigmatism). A means of reshaping the angle distribution within the same cornea is to adjust the distance between mirrors within the galvanometer 106, which have a distance of d2. Persons of ordinary skill in the art will appreciate that the mirrors within an optical scanner may be positioned at an angle, and while the embodiment referenced in FIG. 5 could suggest the mirrors repositioning along a vertical axis, it is not necessary for the mirrors to be disposed along a vertical axis. Regardless, modifying the relative position of the mirrors, either separately or together with the galvanometer's 106 overall position (i.e. d1), is a means of adapting the system to the different corneal curvature deviations within the same cornea, such as astigmatism, by reshaping the angle distribution of the probing beam. Modifying these two aspects of the galvanometer 106 enhances the system's ability to generate self-interference in corneas with curvatures that differ significantly from the average, allowing for compensation for aberrations like astigmatism.

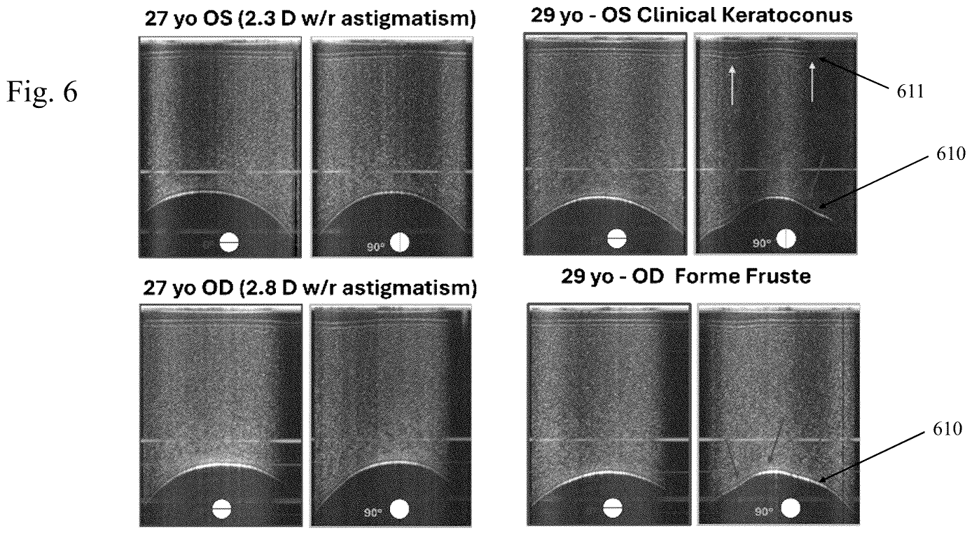

Referring now to FIG. 6, there is shown the results of the self-referencing common-path mode in the left (OS) and right (OD) eyes of both a healthy 27-year-old subject and a 29-year-old subject with keratoconus. For each measurement, (i.e., the left eye of the healthy 27-year-old, the left eye of the 29 year old subject with keratoconus, the right eye of the healthy 27-year-old, etc.) the OCT image of the anterior cornea segment was taken according to the general inventive concepts along the horizontal and vertical meridian. The images reveal distinctive features: both keratoconus and contralateral eyes show posterior surface irregularities along the vertical meridian 610 (i.e., the 90° meridian), regardless of topographic signs, unlike controls where both meridians are unaffected (though the control does have mild astigmatism). This observation of posterior surface irregularities aligns with topographic assessments of keratoconus, which indicate that keratoconus predominantly affects the corneal shape in the vertical direction. In the eye with keratoconus (OS), the vertical image also shows epithelial thickening around the cone 611. Shadows in the OCT images, likely due to increased astigmatism or keratoconus, distort the corneal shape, possibly increasing the angle of the incident beam and reducing collection of back-scattered light.

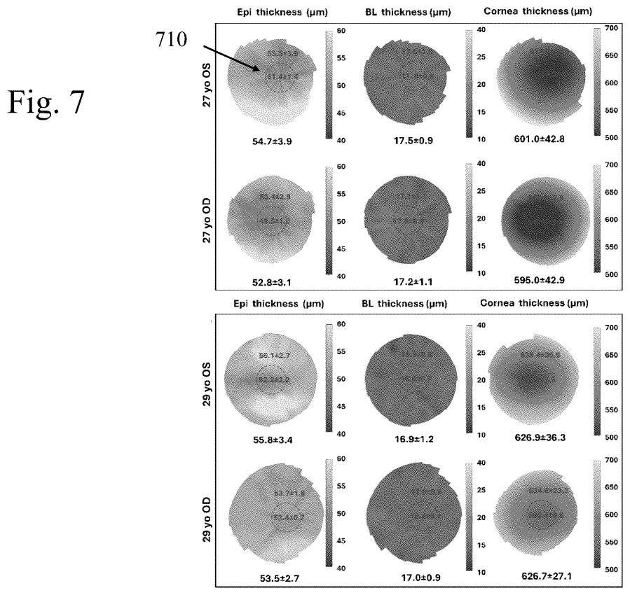

Referring now to FIG. 7, there is shown a map of corneal layer thickness for both subjects. The volumetric OCT images were processed to map the thickness of the anterior corneal layers and the whole cornea. In common-path mode, segmentation of the anterior epithelium is not required as it aligns with the zero-delay line. Other interfaces (Epithelium—Bowman's Layer, Bowman's layers—Stroma and Endothelium—Aqueous) were segmented with a custom-made peak detection algorithm. The thickness was calculated using a group refractive index of 1.387 at 850 nm to convert optical distances into geometrical distances for all layers.

Mean and standard deviation thickness within and outside a 3 mm radius circle centered at the corneal apex 710 are shown. The inventive method was able to visualize how 27-year-old control subject exhibits increased peripheral epithelial thickness in both eyes, possibly due to elevated corneal astigmatism (OS: 2.3 D; OD: 2.8 D). Further, the inventive method was able to visualize that the keratoconus subject shows peripheral epithelial thickening in the affected eye, with moderate differences in the contralateral eye (Forme Fruste), consistent with vertical OCT images. Conventional methods do not allow for measuring the thickness of the layers within the central 1 mm because of the saturation artifacts caused by telecentric scanning. Additionally, the inventive method also provides better visibility of the thin layers in FIG. 6, which aids in facilitating the generation of this map seen in FIG. 7. For example, Bowman's layer thickness is uniform across subjects and zones. The control subject shows no significant inter-eye differences, while the keratoconus subject has central corneal thinning in the affected eye. Mean thicknesses align with typical values reported by conventional OCT imaging.

The following examples illustrate features and/or advantages of the systems, and methods according to the general inventive concepts. The examples are given solely for the purpose of illustration and are not to be construed as limitations of the general inventive concepts, as many variations thereof are possible without departing from the spirit and scope of the general inventive concepts.

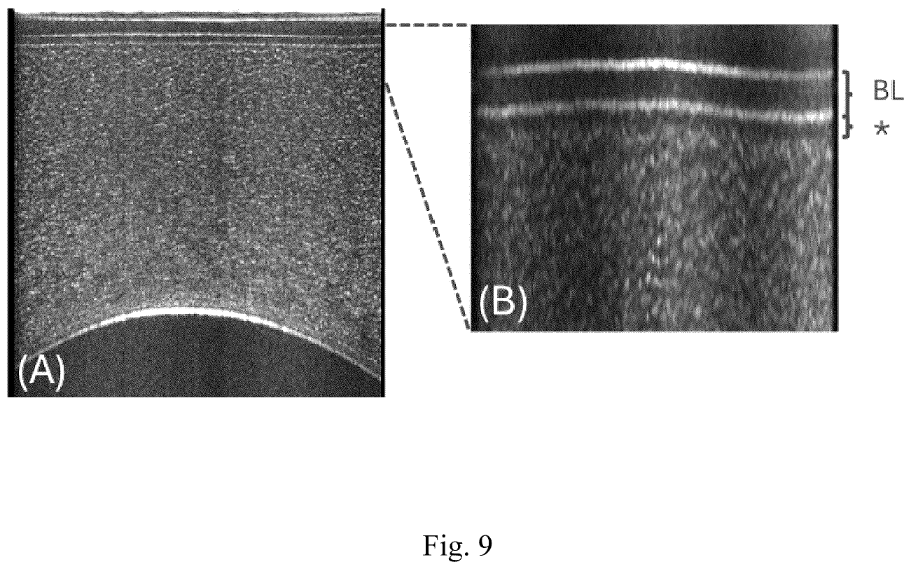

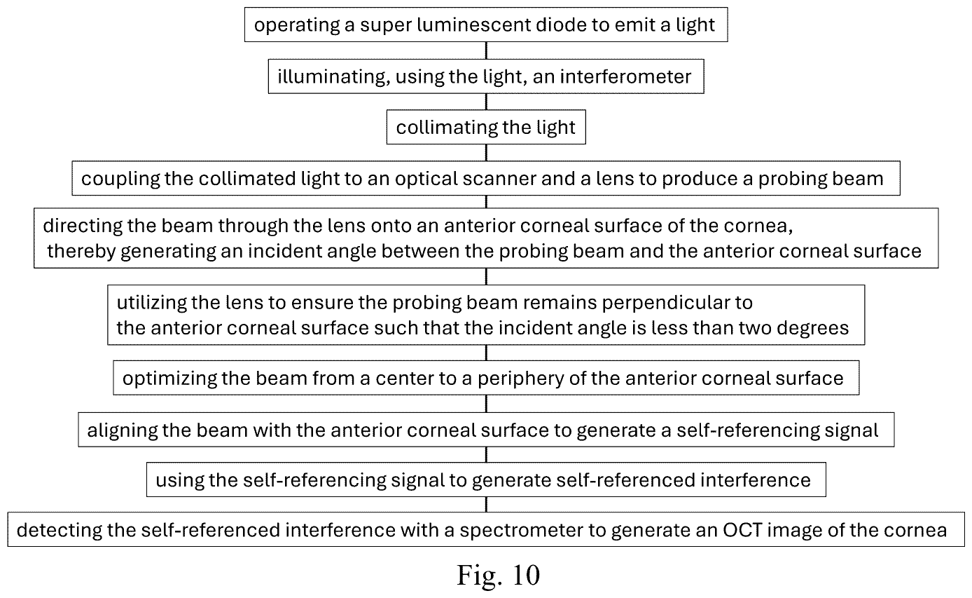

A SD-OCT system (850 nm, 2.7-μm axial resolution) with a common-path interferometer and a custom hypercentric scanning lens was used to maintain near-normal beam incidence to the cornea over a 9 mm diameter (Ruggeri et al., Optics Letters, 2025). This configuration enables visualization of a thin hyporeflective zone beneath Bowman's layer (FIGS. 9A, B) that is not detected with conventional telecentric OCT, likely due to peripheral beam obliquity and defocus induced by corneal curvature and the resulting lateral sensitivity roll-off. Healthy subjects (n =17; ages 23-48) were imaged under IRB-approved protocols. In one eye per subject, 18 radial B-scans (2000 A-lines) were acquired at 10° spacing, and three consecutive meridians were averaged to enhance visibility of the hyporeflective zone. The boundaries of the epithelium, Bowman's layer, and hyporeflective zone were segmented using reflectivity-based boundary detection with smoothing and manual refinement, and two-dimensional thickness maps were reconstructed for all three layers and divided into superior/inferior and central (≤3 mm) and peripheral (3-9 mm) zones.

Mean thickness across subjects (mean ±SD) was 54.8±2.0 μm for the epithelium and 16.4±1.4 μm for Bowman's layer, with both layers remaining spatially uniform across the corneal zones. The hyporeflective zone had an overall mean thickness of 7.8 μm (range: 5.7-10.1 μm), with a low cumulative zonal variability (SD=0.6 μm), which may partially reflect image processing steps (averaging, smoothing, and interpolation) rather than true anatomical uniformity.

This study likely represents the first in vivo OCT-based visualization and quantitative mapping of a thin, low-reflectance layer beneath Bowman's layer across a 9-mm corneal diameter. Its location and morphology suggest possible correspondence with previously described K-structures and motivate further investigation of its role in disease-related structural remodeling.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims.

Parameters identified as “approximate” or “about” a specified value are intended to include both the specified value and values within 10% of the specified value, unless expressly stated otherwise. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

While the invention has been illustrated and described in detail in the drawings and foregoing description, the same is to be considered as illustrative and not restrictive in character. It should be understood that only the exemplary embodiments have been shown and described and that all changes and modifications that come within the spirit of the invention are desired to be protected.

What is claimed is:

1. A method for wide field self-referenced interference OCT imaging of the cornea, comprising:
operating a super luminescent diode to emit a light;
illuminating, using the light, an interferometer;
collimating the light;
coupling the collimated light to an optical scanner and a lens to produce a probing beam;
directing the beam through the lens onto an anterior corneal surface of the cornea, thereby generating an incident angle between the probing beam and the anterior corneal surface;
utilizing the lens to ensure the probing beam remains perpendicular to the anterior corneal surface such that the incident angle is less than two degrees;
optimizing the beam from a center to a periphery of the anterior corneal surface;
aligning the beam with the anterior corneal surface to generate a self-referencing signal;
using the self-referencing signal to generate self-referenced interference; and
detecting the self-referenced interference with a spectrometer to generate an OCT image of the cornea.

2. The method of claim 1, further comprising:
opening an optical shutter on a reference arm of the interferometer such that a mirror is accessible;
using the mirror to generate a preliminary reference signal;
positioning the probing beam to create an incident angle of between two and six degrees, thereby generating a preliminary image;
using the preliminary image to realign the beam such that the incident angle is less than two degrees;
closing the shutter, such that the anterior corneal surface provides the self-referencing signal; and
refining the beam to optimize imaging.

3. The method of claim 1, wherein the lens is a hypercentric lens such that the beam converges at a point behind the cornea.

4. The method of claim 1, wherein the diode emits a light at a wavelength of between 400 nm and 2500 nm.

5. The method of claim 1, wherein the optical scanner comprises a dual axis galvanometer, a MEMS scanner, a polygon scanner, an acousto-optic deflector, an electro-optic scanner, a resonant scanner, or a liquid crystal optical scanner.

6. The method of claim 1, wherein the interferometer comprises a free space interferometer or a fiber-optic coupler.

7. The method of claim 1, wherein the OCT image comprises an imaging depth provided by the spectrometer and the imaging depth is between 0 and 40 mm and at a rate between 1000 A-lines/second and 1,000,000 A-lines/second.

8. The method of claim 1, wherein the method further comprises reshaping an angle distribution of the probing beam incident on the anterior corneal surface.

9. The method of claim 8, wherein the position of the optical scanner is adjusted to reshape the angle distribution of the probing beam.

10. The method of claim 9, wherein the optical scanner includes at least one mirror disposed within, and wherein the position of one or more mirrors disposed within the optical scanner is adjusted to reshape the angle distribution of the probing beam.

11. A method for suppressing corneal apex reflection artifacts in dual-path OCT imaging, comprising:
operating an interferometer in a dual-path mode with an optical shutter disposed in a reference arm in an open state to acquire a first OCT image of the cornea;
closing the optical shutter to disable the reference arm and operate the interferometer in a self-referenced common-path mode;
acquiring a second OCT image comprising self-referenced interference generated at a corneal apex region;
spatially registering the first and second OCT images;
retaining peripheral corneal image data from the first OCT image;
retaining apex corneal image data from the second OCT image; and
combining the retained data into a composite OCT image in which the apex reflection artifact is removed while preserving native corneal curvature.

12. The method of claim 11, wherein the second OCT image is generated when an incident beam is substantially perpendicular to the corneal apex.

13. The method of claim 11, wherein the composite OCT image preserves dual-path axial depth geometry outside the apex region.

14. The method of claim 11, wherein the optical shutter comprises a mechanical shutter, electro-optic modulator, acousto-optic modulator, or MEMS-based optical shutter.

15. The method of claim 11, wherein the first and second OCT images are acquired sequentially with a time gap of less than 200 milliseconds.

16. The method of claim 11, wherein the composite OCT image eliminates vertical saturation artifacts while preserving epithelial, Bowman's layer, and posterior corneal boundary visibility.

17. A hypercentric lens comprising:
a wide field lens configured to allow for a wide field of imaging;
one or more achromatic lenses;
wherein at least one achromatic lens is configured to expand a beam of light passing therethrough such that the beam of light fills an aperture of the wide field lens; and
wherein a distance between the wide field lens and a cornea is less than a distance between the one or more achromatic lenses and the cornea.

18. The hypercentric lens of claim 17, wherein the hypercentric lens has an optimal focus at a working distance wherein the beam of light aligns perpendicularly to the cornea.

19. The hypercentric lens of claim 17, wherein the wide field lens has a surface that is aspheric.
